(19) United States
(12) Patent Application Publication
(10) Pub. No.: US 2007/0027452 A1
(43) Pub. Date: Feb. 1, 2007

(54) Insertion instrument for non-linear medical devices

(76) Inventors: Signe E. Varner, Los Angeles, CA (US); Matthew McCormick, Forest Falls, CA (US); Aaron C. Barnes, Washington, DC (US); Timothy M. Kloke, Chaska, MN (US); Nathan Robert Fox Beeley, Irvine, CA (US); Doug Patton, Irvine, CA (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/436,277

(22) Filed: May 18, 2006

Related U.S. Application Data
(60) Provisional application No. 60/682,454, filed on May 18, 2005.

(51) Int. Cl. A61F 9/00

(52) U.S. Cl. 606/107

(57) ABSTRACT

The invention provides an insertion instrument and methods for rotatably inserting an insertable medical device into a target region of the body. In particular, the instrument can be used in a method for rotatably inserting a medical device having a helical shape into a portion of the eye. The instrument includes a distal portion with a securing member that can hold a portion of the device during the rotational insertion process, and an actuating member that can be triggered to gently release the device upon insertion of the device into the target site.

### CROSS-REFERENCE TO RELATED APPLICATION

The present non-provisional Application claims the benefit of commonly owned provisional U.S. patent application having Ser. No. 60/682,454, filed on May 18, 2005, and entitled Insertion Instrument for Non-Linear Medical Devices.

### FIELD OF THE INVENTION

The invention relates to an instrument and methods for inserting a non-linear medical device into a limited-access region of the body. More specifically, the invention relates to a device and methods for inserting a non-linear drug-delivery device into the vitreous of the eye.

### BACKGROUND OF THE INVENTION

Tools for inserting linear-type medical devices such stents and grafts into the vasculature are well known in the art. Stent insertion has been traditionally performed by crimping a stent onto the insertion element such as catheter, then transporting the stent via the vasculature to a target site. More recently, improvements in this procedure have been seen in stent insertion processes carried out using an insertion tool made from a catheter having a balloon part. Once the stent has been brought to the site of implantation by a catheter, it is possible to deploy the stent by distending the balloon. That is, the stent is brought from its radially contracted condition to its radially expanded or extended condition, in which the stent performs the desired action of stenting on the portion of the vessel being treated. Advances in the art of stent insertion tools have greatly improved these surgical techniques and underscore the importance of providing improvements in the technology of insertion tools for medical devices in general, which ultimately result in greater safety to the patient.

At least in part to the recent success in the use of drug-eluting stents (DES) in percutaneous coronary interventions, the use of drug-eluting implantable medical devices for the local delivery of drugs has received much attention. Drug-eluting implantable medical devices such as DES present or release bioactive agents to their surroundings (for example, luminal walls of coronary arteries). Generally speaking, a bioactive agent can be coupled to the surface of a medical device by surface modification, embedded within and released from within polymeric materials (matrix-type), or surrounded by and released through a carrier (reservoir-type). The polymeric materials in such applications should optimally act as a biologically inert barrier and not induce further inflammation within the body. In other cases, the local delivery of drugs from an implanted medical device may be provided from within the device itself, such as from a reservoir or channel in the device from which the bioactive agent is eluted, rather than from a polymeric coating. In either case, drug-eluting implantable medical devices intended to be delivered to a target site other than vascular sites, such as limited access regions like the eye or the ear, have also received attention for their ability to provide local therapeutic action.

Examples of therapeutic agent delivery devices that are particularly suitable for delivery of a therapeutic agent to limited access regions, such as the vitreous chamber of the eye and inner ear are described in U.S. patent No. U.S. Pat. No. 6,719,750 and U.S. Patent Application Publication No. 2005/0019371 A1. The insertable medical devices described in these patent documents have a non-linear shape, such as a helical or coil shape, and are able to releasably deliver a bioactive agent following insertion into the target site. Insertion of helical or coil shape devices can be performed by screwing or twisting the body member into the eye. These devices can also include a cap portion on the proximal end of the device. During the insertion process, the body member can be screwed or twisted until the cap abuts the outer surface of the eye. The cap can anchor and stabilize the device following insertion, preventing unwanted movement.

The process of inserting these types of small medical devices into portions of the body such as the eye, however, can be rather delicate. Ocular insertion of a device can be performed by rotatably driving the device through the scleral tissue through a penetration in the scleral tissue (trans-scleral insertion) or through a sclerotomy. This process can require gentle techniques, as the scleral tissue is soft and can be disrupted or damaged by mechanically aggressive or crude techniques. Furthermore, overall handling can be challenging due to the relatively small size of the insertable device.

Preferred forms of the rim or cap, as described in U.S. patent No. U.S. Pat. No. 6,719,750 and U.S. Patent Application Publication No. 2005/0019371 A1, include rounded edges, which can minimize irritation to the eye following implantation of the device. While these rounded edges can be beneficial with regard to patient comfort, etc., these types of cap designs are not ideal for the process of insertion of the medical device. Cap configurations that include sharp edges, generally undesirable in methods for the treatment of the eye, can facilitate the application of torque to the cap or head of the device during the insertion process. This presents challenges for methods and the design of tools that can be used to facilitate insertion of medical devices that are rotatably inserted into target portions of the body, such as the eye.

Furthermore, in many cases the helical or coiled portion of the implantable device includes a coating, such as a polymeric coating that is capable of releasing a bioactive agent. In these cases, it is generally desirable to avoid processes that would compromise the integrity of the coating. This introduces restrictions in the way the device can be held for the insertion process.

### SUMMARY

The present invention provides an instrument and methods for inserting a non-linear medical device into a limited access region of the body. In exemplary embodiments, the instrument is used to insert a non-linear medical device into a portion of the eye, such as the vitreous. The instrument is preferably used to promote the rotational insertion of a medical device having a helical or coil shape, into a target site such as the eye.

The insertion instrument includes a proximal portion, which can be held by a user, and a distal portion, which is used near the insertion site. The distal portion includes a member for fastening the insertable medical device to the insertion instrument (i.e., a securing member) during the process of rotatably inserting the insertable medical device into a target site. For insertion of a non-linear medical device, such as those that have a helical or coiled shape, the insertion method includes rotation of the securing member to provide corresponding rotational movement of the insertable medical device that is held in place by the securing member. The securing member on the distal portion of the instrument is arranged to translate sufficient torque and provide stability to the medical device so that it can be inserted into the target site with relative ease and accuracy. Essentially, rotational movement of the distal portion of the insertion instrument drives the insertable medical device into a target site in a screw-like manner.

The insertion instrument can also unobtrusively release the medical device after it has been inserted into the target site. The insertion instrument includes an actuating mechanism that causes the securing member to disengage the insertable medical device. Upon insertion of the device into the target site, the insertion instrument is actuated to cause the release of the device from the securing member.

Given this, in one aspect, the invention provides an insertion instrument for rotatably inserting an insertable medical device into a target site in the body. In some aspects the target site is a portion of the eye. The instrument comprises a proximal portion and a distal portion, the distal portion comprising a securing member capable of engaging an insertable medical device and holding the device in position for rotational insertion into the target site. The instrument also includes an actuating mechanism for releasing the insertable medical device from the securing member.

In some aspects, the insertion instrument is configured so the securing member is rotatable along with the housing of the insertion instrument, which is held by a user. In other aspects, the securing member is independently rotatable from the housing of the instrument. For example, the instrument can include a piston or axle which is in rotatable communication with the securing member, and which is independently rotatable from the housing that is held by the user. This feature can facilitate the insertion process by reducing the overall movement of the instrument.

In many cases, the insertable medical device includes a proximal portion having a head configured to fit within a socket that is formed by the securing member. The head on the insertable medical device can be in any suitable form, such as a rim or cap. Generally, the size of the head is relatively small, and in some aspects has a volume of about 5 mm³ or less. In some aspects the head has a diameter of about 2.5 mm or less, and in some aspects a height of about 0.5 mm or less.

Generally, the securing member is configured to have a shape that can encompass a portion of the proximal end of the device such as the head. When the insertion instrument is actuated to engage and hold the proximal portion of the device, parts of the securing member are brought into contact with the proximal portion. The contact is sufficient to stabilize the entire device for an insertion process.

In some aspects, the securing member comprises two or more radially contractible and expandable fingers. The fingers can define a socket into which a proximal portion of a medical device can be placed and held during an insertion process. Preferably, the fingers are configured to be circumferentially disposed about the proximal portion of the medical device. The proximal portion of the medical device can be released from the socket by triggering the actuating mechanism, which can cause proximal-to-distal movement of the fingers in relation to the distal end of the instrument, thereby allowing radial expansion of the fingers and enlarging the socket to free the distal end of the medical device.

In another aspect of the invention, the insertion tool includes a vacuum member, which allows the insertable medical device to be held in position by suction during the insertion process. The securing member can include a socket, in which the proximal end of the medical device can be seated, and which is in gaseous communication with a vacuum chamber. With the device placed in the socket, the actuating mechanism can be triggered to evacuate the chamber, fastening the device to the proximal end of the instrument. The actuating mechanism can also be triggered to release the vacuum following insertion, thereby causing release of the device. A vacuum mechanism can also be used in combination with, for example, a securing member that also includes radially expandable and contractible fingers which clamp the proximal end of the device in the socket.

When in an engaged position, the securing member of the insertion tool can fasten the head of the insertable medical device. In an engaged position, the distal end of the securing member generally does not extend beyond the distal end of the head of the insertable medical device. This minimizes or eliminates contact between the distal end of the insertion instrument and tissue of the target site and therefore avoids mechanically disrupting or damaging tissue, such as the conjunctival or scleral tissues of the eye. The securing member is designed to be compact and unobtrusive, but can also sufficiently stabilize a device in a manner so that most of, or the entire portion of the device that is not in contact with the securing member can be rotatably inserted into a target site.

The inventive design and function of the insertion instrument provides the needed stability for processes involving the rotational insertion of smaller insertable medical devices. For example, given the more delicate environment of the target tissue such as the eye, the invention provides an insertion tool that is designed to allow the user to stably hold the medical device during insertion into the target site, and when actuated, to gently release the device in a manner that does not disrupt its placement in the tissue of the target site.

Another advantageous feature of the insertion instrument is a distal portion that has a configuration that allows favorable viewing of the target site, including the medical device that is engaged by the securing member. In many aspects the distal portion of the instrument is small and has a tapered shape allowing the user to adequately visualize the target site and the insertion process. This design is also useful in manipulating the insertion instrument, as it is not bulky, and therefore easy to move and rotate during the insertion process.

The present instrument provides many advantages for the rotational insertion of a device into a relatively soft tissue such as scleral tissue, or into viscoelastic body fluid such as the vitreous. In many aspects of the invention, rotational insertion is performed in these types of target tissues or fluids, which are considerably more delicate than other body tissues such as bone tissue.

Use of the insertion tool is also advantageous in aspects wherein a drug delivery coating is provided on the majority of the device (for example, on all or a portion of the surface of the distal portion of the device). Since the securing member engages only the proximal portion of the device, the risk of physical damage to the coating on the distal portion of the device is minimized or eliminated.

In some aspects, the insertion instrument can be provided to a user with the medical device pre-loaded in the securing member of the instrument. Therefore, the invention also contemplates kits that include an insertion instrument and device. A pre-loaded insertion instrument could minimize handling of the device and thereby reduce the likelihood that the device may be improperly positioned within the securing member, or that a portion of the device, such as a polymeric coating, may become damaged. The kits can be provided in packaging and can be sterilized.

Therefore, in another aspect, the invention also provides a kit for rotatably inserting an insertable medical device comprising a non-linear shape into a target site. The kit comprises (a) an insertable medical device comprising a distal portion having a non-linear shape and a proximal portion comprising a head, and (b) an instrument comprising (i) a proximal portion and a distal portion, the distal portion comprising a securing member capable of engaging the head of the insertable medical device and holding the device in position for rotational insertion into the eye, and (ii) an actuating mechanism for releasing the insertable medical device.

### BRIEF DESCRIPTION OF THE DRAWINGS

FIG. 1a is an illustration of an insertable medical device having a helically shaped body member and a head, and which can be used in conjunction with the insertion instrument of the invention. FIGS. 1b-1d are cross sectional views of various embodiments of the head of the insertable medical device.

FIG. 2 shows a cross-sectional schematic view of an eye.

FIG. 3 is perspective view of one embodiment of the insertion instrument of the present invention.

FIG. 4 is an exploded perspective view of a preferred form of the distal portion of the insertion instrument.

FIG. 5 is a cross-sectional illustration of the piston and securing member taken in a plane substantially parallel to the axis of the insertion instrument.

FIG. 6 is an illustration of the securing member viewed from the distal end of the insertion instrument showing an arrangement of four fingers defining a circular socket.

FIGS. 7a and 7b are cross-sectional illustrations of the securing member in a disengaged state, and in an engaged stated with a fastened medical device, respectively, taken in a plane substantially parallel to the axis of the insertion instrument.

FIGS. 8a and 8b are cross-sectional illustrations of another embodiment of the securing member in a disengaged state, and in an engaged stated with a fastened medical device, respectively, taken in a plane substantially parallel to the axis of the insertion instrument.

FIGS. 9a and 9c are cross-sectional illustrations of insertion instruments including an internal mechanism allowing for rotation of the securing member independent of the instrument housing, taken in a plane substantially parallel to the axis of the insertion instrument. FIG. 9b is a cross-sectional illustration of the securing member of the insertion instrument of either 9a or 9b in a disengaged state. FIG. 9d is a perspective view of the insertion instrument including an internal mechanism allowing for rotation of the securing member independent of the instrument housing. FIG. 9e is a perspective view of the piston of the instrument illustrated in FIG. 9d.

FIG. 10 is a perspective view of an insertion instrument including a stabilizing member.

FIGS. 11a and 11b are cross-sectional illustrations of the distal portion of an insertion instruments including a stabilizing member, taken in a plane substantially parallel to the axis of the insertion instrument.

FIG. 12 is perspective view of an insertion instrument including a vacuum mechanism.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention. All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

An “insertion instrument” or “instrument” as used herein refers to a tool used to rotatably insert an implatable medical device into a target site in a subject. An “insertable medical device” or “device” refers to a medical article that can be held by the insertion instrument and inserted into a target site in a subject.

Generally the present invention relates to an insertion instrument for rotatably inserting a small medical device into a patient. The insertion instrument includes a proximal portion, which refers to the portion of the instrument, that when in use, is at the user end (that is, closer to a user than to an insertion site). A user generally holds the proximal portion of the insertion instrument. The distal portion of the insertion instrument includes a securing member, that, when in an engaged state, is able to secure a proximal portion of an insertable medical device, thereby temporarily fastening the insertable medical device to the insertion instrument for the insertion process. Therefore, when in use, the distal portion of the insertion instrument is intended to be located near the insertion site.

The securing member is arranged to securely hold a proximal portion of the device so the device can be rotatably inserted into a target site within the human body. The device can be rotatably inserted into a target site either partially or fully. In one aspect of the invention, the target site is a limited access region of the body such as the eye or the inner ear. In aspects involving insertion into the eye, the device is secured during the rotational insertion, and when the device is sufficiently inserted into the eye, the securing member is actuated to release the device so, for example, the head of the device abuts the scleral tissue on the exterior of the eye.

In order to understand aspects of the insertion instrument and methods of its use to deliver a medical device to a target site, reference is made to the details of an exemplary and preferred insertable medical device that has been described in previous patent documents. However, other insertable medical devices capable of being engaged by the insertion instrument of the present invention can be used in processes for rotational insertion of the medical device into a desired portion of the body.

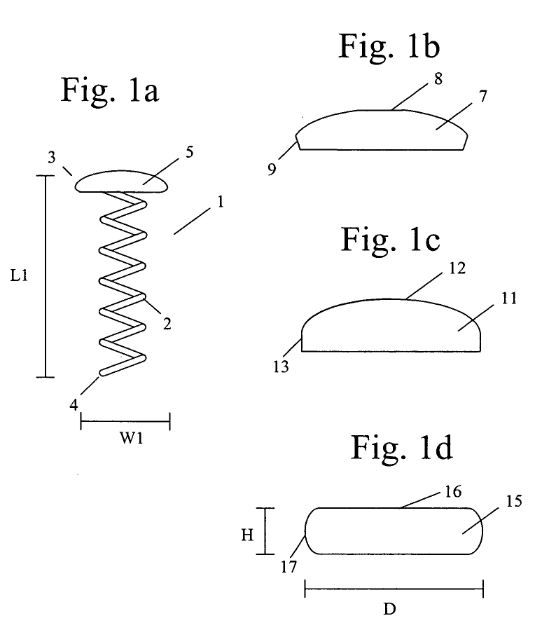

One suitable device that can be inserted into a limited access region of the body is a therapeutic agent delivery device as described in U.S. Pat. No. 6,719,750 (Varner et al.) and U.S. Patent Application Publication No. 2005/0019371 A1. These patent documents describe various non-linear devices that can be inserted into a target site and used to deliver therapeutic agent(s) and/or medicaments from the device. In some embodiments of these documents, the device includes a portion that has a substantially coiled or helical configuration that can be introduced into the target site during insertion. One embodiment of the helically-shaped device of U.S. Pat. No. 6,719,750 is shown in FIG. 1 herein.

As shown in FIG. 1a, and in a preferred aspect of the invention, an insertable medical device 1 that is used in conjunction with the insertion instrument includes a non-linear shaped body member 2, a proximal end 3, and a distal end 4. Preferably, the body member 2 has a substantially coiled or helical shape. The coil shape of the body member allows the device to be screwed or twisted into the target site, such as the eye, through an insertion in a portion of the eye, such as the sclera. The insertion can be approximately the same size as the outer diameter of the body member 2. The distal end 4 of the body member 2 can have a blunt or non-blunt shape. In some embodiments, the distal end has a non-blunt shape and is therefore configured to allow tissue to be pierced during the insertion of the device. For example, the distal end 4 of the device can have a pointed or beveled ramp-like configuration useful for piercing the eye during insertion. In one embodiment, the pointed or beveled ramp-like configuration has a ramp-like angle of about 30°. If the distal end 4 of the body member 2 is used to pierce the eye during insertion, at least the distal end 4 is fabricated of a rigid, non-pliable material suitable for piercing the eye. Such materials are well known and may include, for example, polyimide and similar materials.

FIG. 1a also shows the insertable medical device 1 having a head 5 located at the proximal end 3 of the device 1. The head 5 can be of any suitable configuration and size for insertion of the device and/or placement of the device into a target site. In choosing or forming a head of a particular size and configuration (geometry), the head typically has the inverse geometry of that of the socket of the securing member.

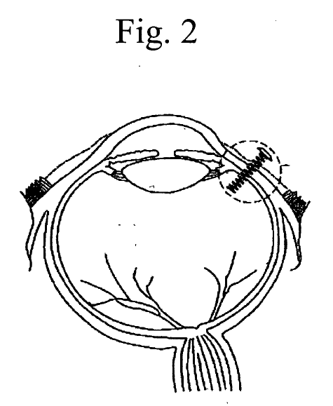

The head can also function to stabilize the device once implanted into a target site, such as the eye (referring to FIG. 2), after being released from the securing member. For example, the device can be inserted into the vitreous of the eye through a penetration or incision in the scleral tissue until the distal face of the head abuts the scleral tissue. If desired, the head may then be sutured to the eye, using an optional one or more holes that can be present in the head, to further stabilize and prevent the device from moving once it is implanted in its desired location.

The overall size and shape of the head is not limited to any particular configuration. In most embodiments, when viewed from the proximal end of the device, the head will have a circular shape, and generally a cap-like shape when viewed in perspective. Referring to FIG. 1a, although the head 5 is shown to have a generally smooth cap-like shape, the head may optionally have a faceted dome-like shape. Alternatively, when viewed from the proximal end of the device, the head may have non-circular shape, for example, a triangular, rectangular, hexagonal, etc., shape. However, to minimize irritation to the eye, the head preferably has a rounded surface.

In some aspects, the head has a cap or rim configuration similar or the same as that shown in FIG. 1b. The head 7 includes a flat top 8 at the proximal end of the medical device, and a straight wall 9 about periphery of the head 7.

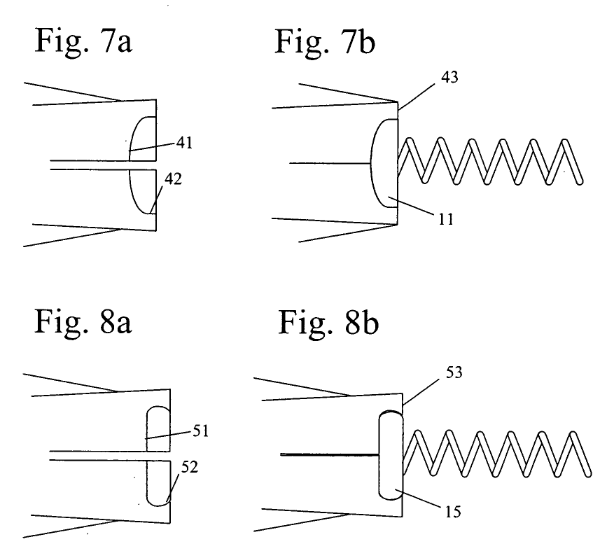

In some aspects, the head has a configuration similar or the same as that shown in FIG. 1c. The head 11 includes a rounded top 12 at the proximal end of the device, and a straight wall 13 about periphery of the head 11. Referring to FIG. 7b, an implantable medical device is illustrated having a head 11 with the configuration as shown in FIG. 1c engaged in the socket of a securing member of an insertion instrument.

In some aspects, the head has configuration similar or the same as that shown in FIG. 1d. The head 15 includes a flat top 16 at the proximal end of the device, and a rounded wall 17 about periphery of the head 15. Referring to FIG. 8b, an implantable medical device is illustrated having a head 15 with the configuration as shown in FIG. 1d engaged in the socket of a securing member of an insertion instrument.

In many aspects, the head of the medical device is small and has a displacement volume of about 5 mm³ or less. In one exemplary design the head has a displacement volume of about 2 mm³ or about 2.5 mm³. In some aspects, for example, referring to FIG. 1d, the head has a diameter (D) of about 2.5 mm or less. In one exemplary design the head has a diameter (D) of about 2.0 mm. In some aspects, the head has a height (H) of about 0.5 mm or less. In one exemplary design the head has a height (H) of about 0.38 mm.

Optionally, the head on the medical device has few or no indentations or recesses, and is therefore substantially smooth. In some aspects, a medical device having a head with this configuration may be preferred, as tissue, which may otherwise in-grow into these indentations or recesses, can be prevented.

In other aspects the head on the medical device has one, and preferably two or more indentations or recesses. The indentations or recesses can be useful for stabilizing the medical device in the securing member, which can be provided by the a securing member having one or more posts configured for insertion in the indentations or recesses (see FIG. 6). Upon insertion of the medical device, the indentations or recesses can be filled in with a sealant to form a substantially smooth surface.

Preferably, the head of the insertable medical device is configured to remain outside the eye and, as such, the head is sized so that it will not pass into the eye through the opening in the eye through which the device is inserted (see FIG. 2). As indicated, the head may further be designed such that it can be secured to the surface surrounding the insertion.

In an engaged state, the head of the insertable medical device can be contacted by portions of the securing member (for example, see FIGS. 7b and 8b). Contact of the securing member with the head can be sufficient to stabilize the entire device for the insertion process. During insertion of the device into a target site, torque can be applied to the head causing rotational movement of the insertable medical device, driving entry of the device into a target site. Furthermore, either the head or the securing member of the insertion instrument can be configured so that they are shaped to provide an optimal fit in an engaged state.

The materials used in fabricating the insertable medical device are not particularly limited. In some embodiments these materials are biocompatible and preferably insoluble in body fluids and tissues the device comes into contact with. Further, it is preferred that the device is fabricated of a material that does not cause irritation to the portion of the eye that it contacts. In some aspects the insertable medical device is fabricated from a metal or alloy. Metals that can be used to fabricate the device include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35N. Other materials include ceramics. The ceramics include, but are not limited to, silicon nitride, silicon carbide, zirconia, and alumina, as well as glass, silica, and sapphire. Polymeric materials can also be used to fabricate the device. Exemplary polymeric material materials can be pliable and include, by way of example, silicone elastomers and rubbers, polyolefins, polyurethanes, acrylates, polycarbonates, polyamides, polyimides, polyesters, and polysulfones.

The non-linear shape of the insertable medical device provides a number of advantages, such as increased surface area providing for favorable release of one or more therapeutic agent(s). In cases where the device is inserted in the eye, it is desirable to maximize surface area while limiting the length of the device in order to avoid the distal end of the device from entering the central visual field, which may result in blind spots in the patient's vision and increase the risk of damage to the retina tissue and lens capsule. For example, when the device is inserted at the pars plana, the distance from the insertion site on the pars plana to the central visual field is about 1 cm.

A device having a non-linear shape also provides a built-in anchoring system that can reduce unwanted movement of the device and/or unwanted ejection of the device from a target site. For example, the non-linear shape of the device requires manipulation in order for it to be removed from a target site (e.g., a coil-shaped device would require twisting the device out of the eye).

The dimensions and configurations of the insertable medical device can depend on the application of the device. When a device such as shown in FIG. 1a is used to deliver substances to the posterior chamber of the eye, the device is preferably designed for insertion through a small incision that requires few or no sutures for scleral closure, after the insertion procedure has be completed. As such, the device is preferably inserted through an incision that is no more than about 1 mm in cross-section, for example, ranging from about 0.25 mm to about 1 mm in diameter, more preferably less than 0.5 mm in diameter. Accordingly, the cross-section of the tube or wire forming the body member 2 is preferably no more than about 1 mm in diameter, with a preferred range from about 0.25 mm to about 1 mm in diameter. More preferably, the cross section is no greater than 0.5 mm in diameter. As shown in FIG. 1a, the non-linear body member 2 is cylindrical in shape, with a circular cross-section. However, the shape of the body member is not limited and, for example, may alternatively have square, rectangular, octagonal or other cross-sectional shapes. If the material (such as a tube or wire) forming the body member 2 is not cylindrical, the largest dimension of the cross section can be used to approximate the diameter.

When used to deliver agents to the posterior chamber of the eye, the body member 2 (referring to FIG. 1a) has a length (L1) from its proximal end to its distal end 6. The length (L1) can be less than about 1.5 cm, or less than 1.0 cm, and preferably in the range from about 0.25 cm to about 1.0 cm. The length (L1) can be such that when the distal portion of the head 5 abuts the outer surface of the eye, the proximal portion of the body member is positioned within the posterior chamber of the eye.

Thus, in some specific aspects, the invention provides an insertion instrument and methods for rotatably inserting a non-linear shaped medical device into a target tissue. The device has a proximal portion including a head, and a body member with a proximal to distal length (L1) of about 1.5 cm or less, or about 1.0 cm or less, and preferably in the range from about 0.25 cm to about 1.0 cm. Thus, in more specific aspects, the device has a width (W1) of about 0.5 cm or less, and preferably has a width in the range from about 0.2 cm to about 0.5 cm.

The insertion instrument of the present invention can facilitate the rotational insertion of an insertable medical device into a target location in the body. In many aspects, the insertion tool includes features that improve aspects of the insertion process. In the context of the present invention, these features can be used individually or in combination with other features. Combination of selected features can provide added benefits to the fundamental features of the insertion tool, the benefits of which will be seen by the skilled user of the device. These features can improve aspects related to the precision of the insertion process, ease of insertion of the medical device, and safety of the insertion process. While the device is described as an insertion instrument, it can be used just as well as an instrument for the removal of a medical device from a target site after a period of implantation.

In one embodiment, the insertion instrument includes a proximal portion and a distal portion. The distal portion of the insertion instrument has a housing having an inner bore and a piston (e.g., a shaft) slidably disposed within.

In this embodiment, the distal end of the piston can be extended beyond the distal end of the bore. The distal portion of the piston includes a securing member comprising a plurality of radially expandable and contractible fingers. The plurality of fingers can define a socket into which a proximal portion of a medical device can be placed and held during an insertion process. In many aspects, the fingers are configured to circumferentially encompass the periphery of the proximal portion of the medical device.

The securing member is capable of both engaging (holding) and disengaging (releasing) the proximal portion of a medical device in a manner suitable for the rotational insertion and placement of a medical device into a target tissue, such as the eye. By engaging the proximal portion of a device, the entire device is substantially stabilized by the insertion instrument.

In these aspects, the securing member of the insertion instrument is generally is arranged to function in a manner similar to that of a collet. In the engaged position, the inner wall of the housing compresses the fingers radially inward, and the internal dimensions of the socket are reduced. When the proximal portion of the device is placed in the socket and the securing member is in the engaged state, the fingers exert pressure around the periphery of the device and secure the device within the socket of the securing member, sufficient for the rotational insertion of the device into a target site.

After the device has been sufficiently inserted into a target site, the insertion instrument can be actuated causing the securing member to be shifted from an engaged to a disengaged state. For example, in some aspects, the securing member is extended distally in relation to the distal end of the housing. Upon extension, the fingers expand radially outward. Essentially, the proximal end of the device becomes “unsecured,” as the fingers expand radially outward from the central axis. When in a disengaged state, the proximal end of the inserted device can be gently separated from the securing member.

In one configuration of the present invention, the insertion instrument includes a distal portion that is tapered. Preferably, the insertion instrument has a distal portion with an outer diameter that gradually becomes smaller towards the distal end. The distal end can therefore have a conical or pointed shape, which can be very useful for visualizing the insertion processes described herein. The distal portion having the securing member provides adequate strength and stability for holding a device in place during an insertion process.

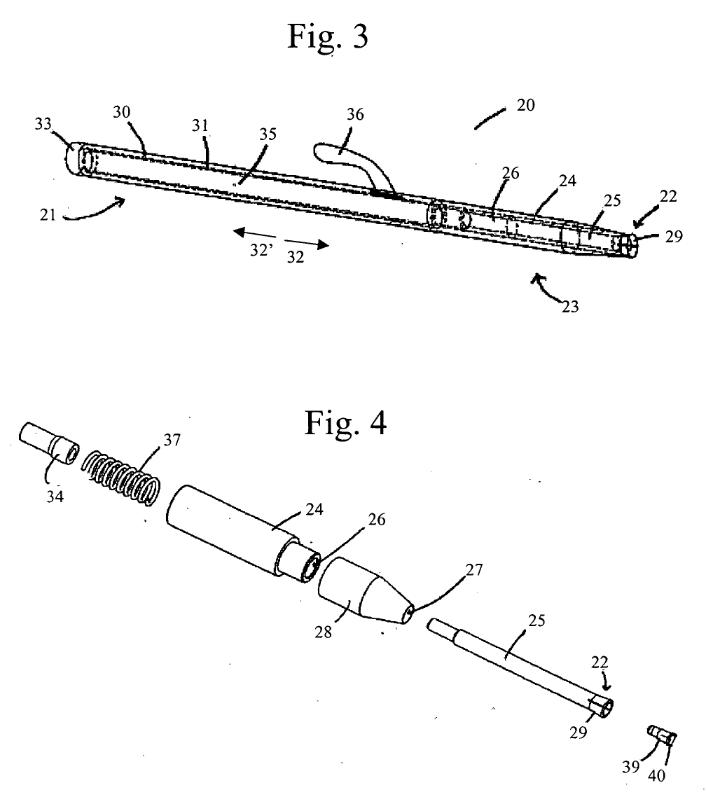

In one embodiment of the invention, as shown in FIG. 3, the insertion instrument 20 includes a proximal portion (user end) 21 and a distal portion 23. In some embodiments, as shown in FIG. 3, the proximal portion 21 is configured to be manually held and operated by a user, such as a surgeon or other individual performing the insertion process. Therefore, the proximal portion 21 of the insertion instrument 20 can be configured as a handle having any shape suitable for performing an insertion process. The handle can have a simple cylindrical shape or, alternatively, can have a shape that is designed to ergonomically fit portions of the user's hand. For example, the handle can have raised portions that allow the user to have a better grip of the device using fingers. The handle can also have a textured or patterned surface to reduce slippage or increase frictional forces between the user and the instrument. This sort of surface can be useful if the user is wearing a hand covering, such as latex gloves.

The proximal portion 21 can be fabricated from any suitable material, including plastics, composites, ceramics, metals, and metal alloys. In some cases it is preferred to use a material that can be readily sterilized, for example, by heat and/or pressure sterilization, such as autoclaving, or by irradiation, such as gamma irradiation, or by chemical sterilization, such as ethylene oxide sterilization. In other cases the handle can be disposable by fabricated all or parts of handle using plastic materials, such as ABS, Teflon™, and Delrin™. The handle can be of any suitable length and outer diameter for use. For example, a handle having a length in the range of about 10 cm or 11 cm can be particularly useful when utilized in methods for rotatably inserting of a device into a target site.

Alternatively, the distal portion 23, or both proximal portion 21 and distal portion 23 of the insertion instrument 20 can be configured for attachment to a support unit (not shown). A support unit can serve for steadying the insertion instrument 20 if it is desired to minimize or eliminate movement associated with insertion of a device as would be performed by hand. For example, the proximal portion 21 of the insertion instrument 20 can be attached to an arm of a support unit that can be adjusted to bring the distal portion 23 of the insertion instrument 20 (along with the attached device) into proximity with the insertion site on the eye. The entire insertion instrument 20, or in some cases the distal portion 23 of the insertion instrument 20, can then be rotated manually or automatically (as discussed below) to provide for rotational insertion of the device into the insertion site of a target site, such as the eye.

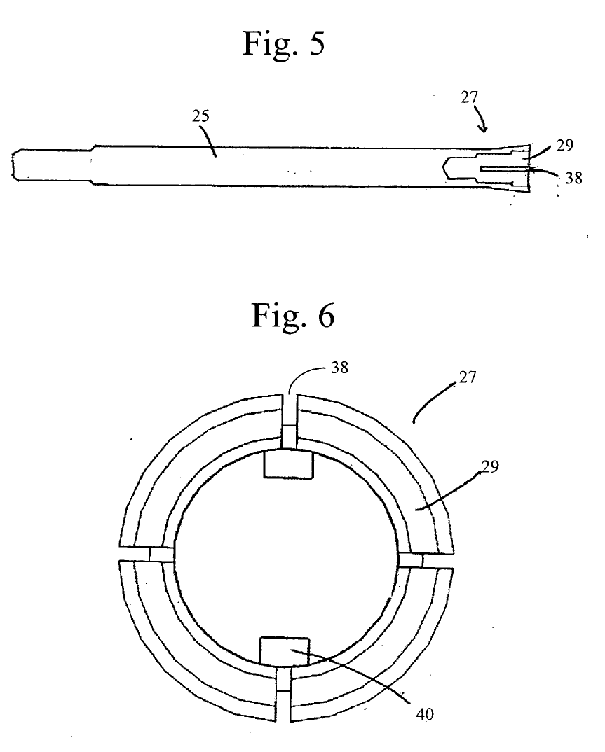

Referring to FIGS. 3 and 4, the distal portion 23 of the insertion instrument includes housing 24 and piston 25 slidably disposed within a bore 26 of the housing 24. The piston can include a securing member 22 at its distal end. (In FIG. 4, the distal portion of the insertion instrument is shown as including two portions: housing 24 and the most distal portion of the housing, the housing nozzle 28. In order to demonstrate some embodiments of the invention, reference is made to FIG. 4, wherein the distal portion includes the housing 24 and housing nozzle 28.) The securing member 22 includes a plurality of fingers 29 (shown in greater detail in FIG. 5) that are used to secure a device for an insertion procedure.

The proximal portion 21 of the insertion instrument 20 can include an actuating mechanism that allows the user to cause the securing member 22 to be in an engaged position. In some embodiments, when the securing member 22 is in an engaged position it is located at least partially within the bore 27 of the housing nozzle 28. That is, the actuating mechanism can cause the securing member 22 to be retracted at least partially into the bore 27 of the housing nozzle 28, forcing the fingers 29 of the securing member 22 into a radially contracted position. In this case, a proximal portion of a device can be placed into the socket of the securing member 22 to securely hold the device when the securing member 22 is in an engaged position.

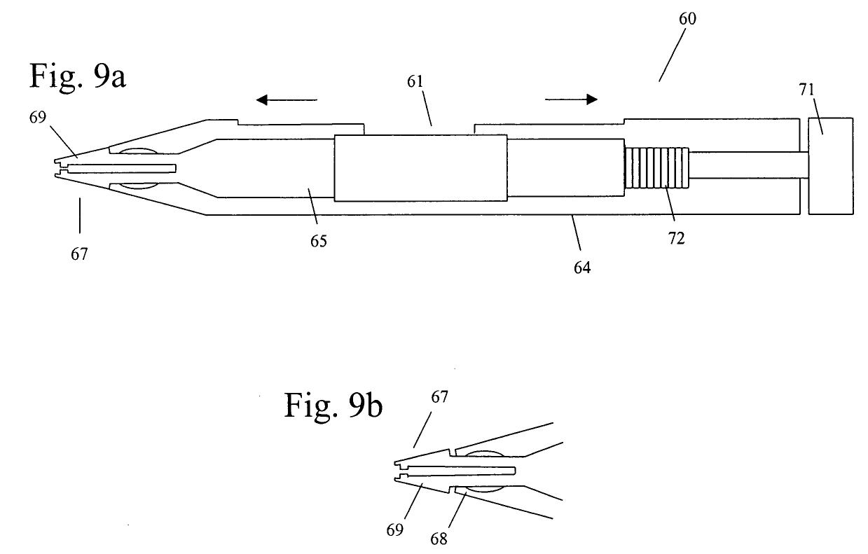

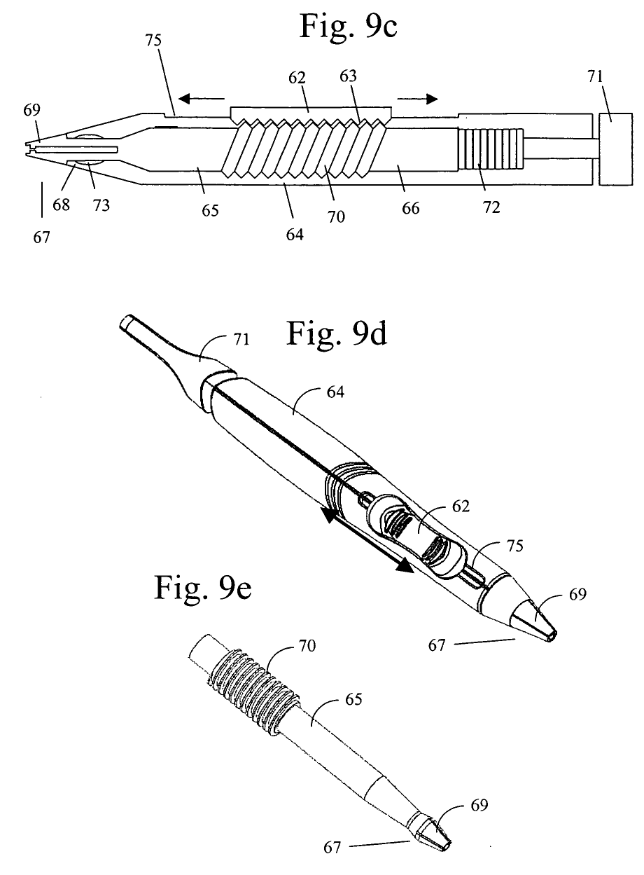

In other embodiments, referring to FIGS. 9a-9e, a portion of the securing member is located beyond the distal end of the housing nozzle 68. The distal portion of the fingers 69, which include the socket, do not enter the bore of the housing nozzle 68 in an engaged state. However, a proximal portion of the fingers 69 are disposed within the bore of the housing nozzle 68, and are configured to cause the radial contraction or expansion of the distal end of the fingers 69 when moved in a proximal direction or distal direction, respectively.

Referring back to FIGS. 3 and 4, the actuating mechanism can also cause the securing member 22 to be extended distally from the bore 27 of the housing nozzle 28 to a disengaged position, wherein the fingers 29 expanded radially upon distal movement of the securing member 22. In a disengaged position, the fingers 29 do not constrain the head of the device and the device can be released from the socket of the securing member 22.

Any sort of actuating mechanism can be used to cause the movement of the piston 25 and securing member 22 in relation to the position of the bore 27 of the housing nozzle 28. However, to demonstrate aspects of the function of one embodiment of the insertion instrument 20 the following description is provided.

In one embodiment, the actuating mechanism is a part of the proximal portion 21 of the insertion instrument 20. Referring to FIG. 3, the proximal portion 21 of the insertion instrument 20 includes a substantially cylindrically-shaped handle 30 having a substantially cylindrically-shaped hollow inner bore 31. The handle 30 can be attached to the housing 24 by screwing the proximal portion of the housing 24 onto the distal portion of the handle 30. Alternatively, the handle 30 and housing 24 can be a unitary piece.

Referring to FIG. 3, a second piston 35 is slidably disposed within the inner bore 31 of the handle 30. Movement of the second piston 35 in a proximal-to-distal direction, as indicated by arrow 32, respectively, and a distal-to-proximal direction, as indicated by arrow 32′, can be caused by actuating a trigger 36 disposed on the outer surface of the handle 30. The trigger 36 can be actuated to cause movement of the second piston 35, which forces movement of piston 25. This movement can cause the distal end of the piston 25 to extend beyond the distal end of the housing nozzle 28. The extension of the distal end of the piston 25 causes the securing member 22 to move distally in direction 32 to a disengaged position.

A set nut 33 can be provided on the proximal end of the handle 30 to serve as an adjustable stop for the second piston 35 so that one may adjust the travel of the second piston 35 within the inner bore 31 of the handle 30. This can allow one to adjust the movement of piston 25, and therefore the distal travel of the securing member 22 in relation to the distal end of the housing nozzle 28. A handle having an actuating mechanism such as this can be obtained from Rumex International Co. (Product ID 12-001 T)

The proximal portion 21, including the handle 30, and the distal portion 23, including the housing 24, can be connected to each other in any suitable manner. For example, the handle 30 and housing 24 can be threaded in order to be screwed into each other. This allows one to change the type of handle 30 that is attached to the distal portion 23, or vice versa. The proximal portion 21 and distal portion 23 of the instrument can be connected so that the actuating mechanism drives movement of the piston 25 in direction 32 resulting in corresponding movement of piston 25 and securing member 22 in direction 32.

In a preferred aspect, the distal portion 23 has a tapered configuration (shown as the distal portion of the housing 24 in FIG. 3, or the distal portion of the housing nozzle 28 of FIG. 4) as it can allow the user to better visualize the insertion process. That is, by having a distal portion 23 with a tapered configuration, the user can observe the insertion site without significant obstruction from the distal portion 23 of the device. Referring to FIG. 4, in some specific aspects, the distal end of the housing nozzle 28 tapers to an outer diameter of about 3 mm. In other specific aspects the non-tapered portion of the distal portion 23 of the insertion instrument 20 (for example, housing 24 of FIG. 4) has an outer diameter of about 6.4 mm. Preferably the distal end of the housing nozzle 28 is constructed from a strong or hardened material, such as stainless steel.

FIGS. 9a-9c also show an insertion instrument having a tapered distal end.

Referring to FIG. 4, and as discussed, the distal portion 23 of the insertion instrument 20 can be formed by attaching housing nozzle 28 having a hollow inner bore 27 to a housing 24 having a hollow inner bore 26. This two-piece arrangement may be useful if it is desired to use a housing nozzle 28 having a different sized bore and a corresponding securing member 22 that is sized to fit the bore. The housing nozzle 28 can be attached to the housing 24 in any suitable manner, for example, by screwing the housing nozzle 28 onto the housing 24. In this case, the bore 26 and bore 27 are continuous and define a hollow inner portion of the distal portion 23 of the insertion instrument 20.

Within the bores 26 and 27 of the housing nozzle 28 and housing 24 is slidably disposed piston 25 having a proximal end and a distal end, where on the distal end of the piston is formed a securing member 22. The proximal end of the piston 25 can be connected to a nut 34 which fits within the bore 26 of the housing 24. The proximal end of the piston 25 and the nut 34 can have threads so that they can be connected and unconnected as desired.

In an alternative aspect, the insertion instrument is configured so the distal end of the housing is movable in relation to the piston (shaft) having a distal portion with a securing member. In this arrangement, the fingers are also radially contractible and expandable, depending on the position of the distal end of the housing in relation to the distal end of the securing member.

Preferably, the piston 25 and the nut 34 are connected within the bore 26 of the housing 24 and are self-retracting. That is, insertion instrument 20 can be actuated to drive second piston 35 in direction 32, thereby forcing piston 25 and the nut 34 to be driven in direction 32 also. However, the piston 25 and the nut 34 can retract into the original position when pressure from the second piston 35 is released. The self-retracting feature can be achieved by spring-loading the piston 25 and the nut 34. For example, and as shown in FIG. 4, a spring 37 can be placed within the hollow inner bore 26 of housing 24.

In order to achieve this configuration, a proximal portion of the housing 24 can have a bore that is larger in diameter than the bore of the distal portion of the housing 24. The bore of the proximal portion has a diameter which accommodates a spring 37 and the nut 34. The piston 25 can pass through the inner diameter of the spring 37 and be attached to the nut 34. Therefore, when piston 25 is actuated and driven in direction 32 to force nut 34 in direction 32, the nut 34 compresses the spring 37, and the securing member 22 is moved in direction 32 to a disengaged position. When pressure from the second piston 35 is withdrawn, the spring 37 causes the retraction of the nut 34 and the piston 25/securing member 22 back in direction 32′.

The securing member 22 can be formed on the distal end of piston 25. The securing member 22 includes two or more fingers 29 that can be extend distally in relation to the distal end of the instrument. The securing member can have any number of fingers more than two, but preferably, the securing member 22 has four or six fingers, and most preferably four fingers. The fingers 29 form the periphery of the socket of the securing member 22, and contact the proximal portion of the device (for example, head 10) when the securing member 22 is in an engaged position.

In some embodiments, and referring to FIG. 5, the piston 25 having the securing member 22 at its distal end has an outer diameter that is generally smaller towards its proximal end (where it is threaded into the nut 34) and larger at its distal end, wherein the securing member 22 is formed. That is, near the distal end of the piston 25, the outer diameter gradually increases to form a bell-shaped distal end.

In order to demonstrate features of one design of the securing member 22, an exemplary process for its fabrication is described. Referring to FIG. 5, one method for fabricating the securing member 22 is to create a bore, or a series or bores, in the distal end of the piston 25, thereby forming at least a portion of the socket of the securing member 22. Preferably, the bore or series of bores is made in a piston having a bell-shaped distal end. For example, a series of progressively smaller bores can be created in the distal end. The extent of the boring will define the thickness of the lateral walls (fingers 29) of the securing member 22.

After distal end of the piston 25 has be bored out to define the socket of the securing member 22, fingers 29 which define the outer walls of the socket can be formed by cutting axial slits 38 in the distal end of the bored-out piston 25. The number of axial slits 38 (slits that are parallel to the axis of the piston 25) that are made in the bored-out end of the piston 25 can define the number of fingers 29 present in the securing member 22. For ease of fabrication, it may be preferred to make an even number of axial slits 38 in the distal end to define two, four, six, or more fingers 29. The securing member preferably has four or six fingers 29. The depth (in a proximal to distal direction) of the axial slits 38 cut into the bored-out distal end of the piston 25 can also define the general length of the fingers 29. In one exemplary embodiment the slits are cut about 2.8 mm into the distal end of the bored-out piston. The width of the cuts is preferably in the range of about 0.25 mm to about 0.5 mm. FIG. 6 shows a view of the securing member 22 and fingers 29 from the distal end of the insertion instrument.

The fingers 29 of the securing member 22 are resilient and are able to expand radially outward when the securing member 22 is in a disengaged position to release the proximal end of a device that is inserted into a target site. Accordingly, the fingers 29 will also be able to contract radially inward when the securing member is in an engaged position.

Referring back to FIG. 4, in order to form the securing member, an insert 39 can be placed into the bore that is formed in the distal end of the piston 25. In some aspects, the insert 39 can be fabricated to include one or more distally-facing tabs 40 (also shown in FIG. 6) that contact, and are preferably insertable into, the head of the device that is held by the securing member. The insert 39 can also be formed to have a concave shape to match that of the shape of a head with a cap configuration. The tabs 40 can be useful for providing torque to the head when rotational force is applied to the insertion instrument and for stabilizing the device when engaged with the securing member. When the insert 39 is seated in the bored-out piston 25, the distally-facing portion of the insert 39 defines the proximal end of the socket.

When the securing member 22 is moved in direction 32′ the outer portions of the fingers 29 contact the inner walls of the bore 27 of the housing nozzle 28 and radially compress the fingers 29, exerting pressure around the periphery of the head of the device. When the securing member 22 is extended from the housing nozzle 28 to a disengaged position, the fingers 29 radially expand, and the head of the device becomes unsecured.

Each finger 29 has a distal portion and a proximal portion. The proximal portion of the finger is connected to, or integral with, the body of the piston (as shown in FIG. 5). In some embodiments, the fingers are at an angled in relation to the axis of the piston. In one exemplary embodiment, the fingers are at an angle of about 6.75° from the axis of the piston. In some modes of practice the fingers 29 an exemplary length of about 2.4 mm.

The fingers can be of various shapes to form a socket with a desired geometry. Various socket geometries are contemplated, and a particular socket geometry can be chosen to according to the particular shape of the proximal end (e.g., head) of the insertable medical device.

A cross sectional view of the geometry of one exemplary socket of a securing member wherein the fingers are in a radially expanded state is shown in FIG. 7a. The cross section illustrates that the socket includes a proximal surface 41 (i.e., closest to the proximal end of the device) having a concave shape and peripheral surface 42 having at least a portion that is straight. FIG. 7b shows the securing member of FIG. 7a with the head 11 of an insertable medical device engaged in the securing member wherein the fingers are in a radially constricted state. The insertable medical device has a head 11 that corresponds to the geometry of the socket, and the socket generally encompasses the proximal and peripheral surfaces of the head 11. As shown in FIG. 7b, the distal ends of the fingers 43 do not extend distally beyond the distal end of the cap.

A cross sectional view of the geometry of another exemplary socket of a securing member is shown in FIG. 8a. The cross section illustrates that the socket includes a proximal surface 51 that and a peripheral surface 52 that is curved. FIG. 8b shows the securing member of FIG. 8a with a head 15 engaged in the securing member wherein the fingers are in a radially constricted state. In this aspect, the fingers 53 provide greater contact with the surface of the head, owing to curved peripheral surfaces (which form lips) on the distal ends of the fingers 53. FIG. 8b also shows that the distal ends of the fingers 53 do not extend distally beyond the distal end of the cap 15.

In another aspect of the invention the insertion instrument is designed to allow rotation of the securing member independently from the housing held by the user. That is, during the process of rotational insertion, rather than rotating the entire insertion instrument to cause corresponding rotation of the insertable medical device that is fastened to the distal end of the instrument, the instrument can be maintained in a (rotational) stationary position and a rotational mechanism can be actuated to provide corresponding rotation of the securing member and engaged medical device. The rotational mechanism can be actuated manually to provide corresponding rotation of the securing member. Optionally, the rotational mechanism can be driven by a motor that is coupled to the securing member.

To illustrate this aspect of the invention, reference is made to the instrument 60 shown in FIG. 9a. The instrument 60 includes a housing 64 and a piston 65 disposed within the housing 64. The piston 65 is independently rotatable from the housing 64 and rotatably connected to the securing member 67. In this regard, the piston can function as, for example an axle or a mandrel, being independently rotatable within the housing 64. In some aspects the piston 65 and the securing member 67 can be formed from the same component. In other aspects, the piston and securing member can be formed from two or more components. For example, the securing member can be configured for connection and removal from the piston.

In a fundamental form, the housing 64 includes an opening 61 allowing direct (manual) access to the piston 65. This allows the user to manually rotate the piston 65 through the opening (for example, by contacting the surface of the piston with a free finger), while holding the instrument in place. The surface of the piston 65 accessible through the opening 61 can be textured to facilitate rotation of the piston 65. For example, the surface of the piston can have a beveled surface.

In another aspect, as illustrated in FIGS. 9c (a cross sectional view of the insertion instrument) and 9d (a perspective view of the insertion instrument), the opening (of FIG. 9a) can be replaced by a sliding member 62 that is in mechanical communication with the piston 65. Sliding member 62 is movable in proximal and distal directions along track 75. Movement of the sliding member 62 can cause rotation of the piston 65 and corresponding rotation of the securing member 67. For example, referring to FIG. 9c, the sliding member 62 can have an inner surface that includes threads 63, and the surface of the piston 65 also includes threads 70. The threads 63 of the sliding member 62 and the threads 70 of the piston 65 are meshed, so that movement of the sliding member 62 causes corresponding rotational movement of the piston 65 and the securing member 67.

FIG. 9e illustrates the piston 65 having threads 70 and the securing member 67 with fingers 69 at the distal end of the piston 65.

Referring to FIG. 9c, the insertion instrument 60 includes an actuating mechanism, which can cause the radial expansion and contraction of the fingers 69. The actuating mechanism includes a knob 71 that is located at the proximal end of the instrument 60, which can be depressed to cause proximal to distal movement of the piston 65 and the securing member 67. The piston 65 can be self-retracting and can be spring-loaded by including spring 72 within the bore of the housing.

Referring to FIGS. 9c and 9d, the distal end of the device includes the securing member 67 with socket for engaging the distal portion of the insertable medical device. Outer portions of the fingers can be supported by bearings 73, which facilitate rotation of the piston 65 and securing member 67. In an engaged position, the housing nozzle 68 of the housing forces the fingers 69 into a radially constricted position. When the piston is extended distally, as shown in FIG. 9b, the securing member 67 is also extended from the distal end of the instrument. When distally extended, the fingers 69 of the securing member 67 expand radially to the disengaged position. In the disengaged position, the socket of the securing member enlarges to a size sufficient for release of the head of the insertable medical device. FIG. 9b shows the distal end of the instrument with the securing member 67 in a disengaged state, the fingers 69 of the securing member 67 being radially expanded, allowing for release of an implantable medical device.

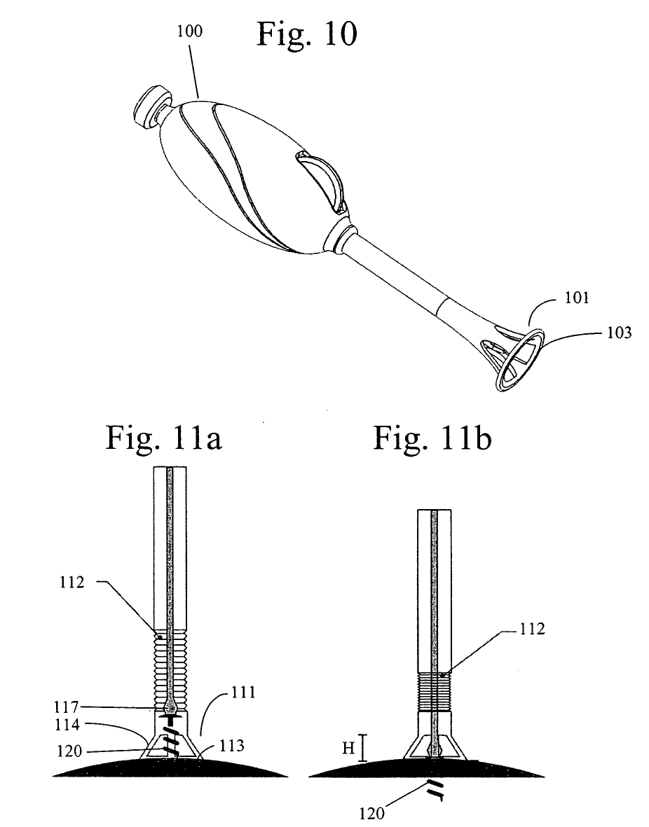

Referring to FIG. 10, in another embodiment, an insertion instrument 100 can also include a stabilizing member 101 on its distal end. A stabilizing member can be used to facilitate insertion of the insertable medical device at the target site by steadying the distal end of the insertion instrument during the insertion process, thereby minimizing unwanted movement.

Referring to FIGS. 11a and 11b the stabilizing member 111 is generally used in combination with a feature that allows the securing member 117 to be moved distally during the process of rotational insertion. This feature can be a collapsible portion 112 of the housing of the distal end of the instrument.

As shown in FIGS. 11a and 11b, the stabilizing member 111 can have a base 113, which represents the most distal portion of the instrument and which is placed in contact with an area about the insertion site. Referring back to FIG. 10, the base 103 is shown as a unitary structure having a circular shape. In one exemplary design the base has a diameter of about 1 cm. A circular (or near circular) shape can be particularly suitable for contacting an outer portion of the eye in order to stabilize the distal portion of the device. However, other shapes, including oval and polygonal shapes can also be suitable for the base. In some aspects, the stabilizing member comprises an optically clear material for favorable visualization of the implantation site. The stabilizing member can also have one or more fenestrations that can also allow favorable visualization.

Referring back to FIGS. 11a and 11b, the base 113 is connected to the insertion instrument at a location generally other than securing member via one or more arms 114, and preferably two or more arms. For example, as shown in FIG. 11a, the arms 114 are connected to a distal portion of the instrument. In preferred aspects, and as shown in FIGS. 11a and 11b, the arms extend from a distal portion of the instrument at an angle from the central axis of the device.

The height (H) of the securing member typically depends on the overall length of the insertable medical article. Generally, the height (H) is not less than the overall length of the insertable medical article. In one exemplary design the height (H) is not less than about 0.5 cm.

Referring to FIG. 11b, the collapsible portion 112 of the housing of the distal end of the instrument is shown in a collapsed state with the medical device being inserted into the target site.

In another aspect of the invention, the insertion instrument includes a vacuum mechanism for securing the insertable medical device to the distal end of the instrument. In this embodiment the vacuum mechanism partially or fully fastens the proximal end of the medical device to the securing member by suction. If the vacuum mechanism partially fastens the device, it can be used in combination with, for example, a securing member that also includes radially expandable and contractible fingers that clamp the proximal end of the device in the socket.

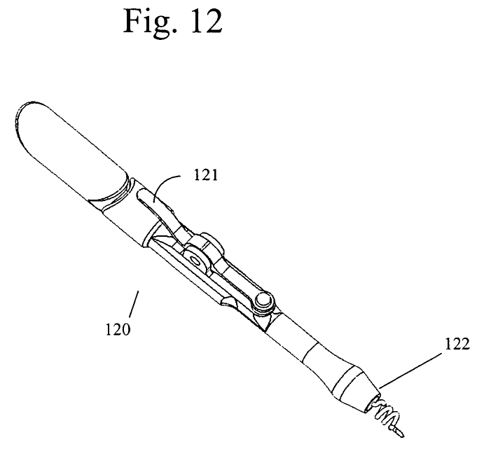

Referring to FIG. 12, an insertion instrument 120 having a vacuum mechanism is shown. The insertion instrument includes a housing, and a bore within the housing that includes a vacuum chamber. Air in the vacuum chamber can be evacuated by triggering the actuating mechanism, which includes lever 121.

In a fundamental form, an instrument that includes a vacuum chamber can include a securing member that is in gaseous communication with the vacuum chamber, the securing member providing a seat, or in some cases, a socket, for the proximal end (e.g. head) of the insertable medical device. As shown in FIG. 12, the securing member 122 includes a socket and a ring gasket located at the proximal portion of the socket. The ring gasket can be made of any suitable elastomeric material, and provides a seal to ensure that the vacuum is maintained when the device is seated in the socket. In the center of the ring gasket a bore is in gaseous communication with the vacuum chamber.

The insertion instrument of the present invention can be used in a method for rotatably inserting a medical device into a target site. Some aspects of the invention are related to methods for the rotatable insertion of a device into a viscoelastic fluid or non-osseous tissue. In exemplary modes of practice, the insertion instrument is used for rotatably inserting an insertable medical device into a portion of the eye.

The insertion instrument can be used to rotatably insert the medical device into to a supple body tissue and/or body compartment that includes a gel-like biological material. For example the device can be delivered to a non-osseous tissue or body compartment that includes non-osseous biological material. “Non-osseous” refers to tissue or biological material that is not connective tissue having a matrix of which consists of collagen fibers and deposited calcium salts in the form of an apatite. The process of inserting a device into a non-osseous tissue can be more delicate than the process of inserting a device such as a bone screw into bone or other types of tissues that have been hardened by calcification.

In exemplary embodiments, the insertion instrument is used to provide a rotatably insertable medical device to a portion of the eye. Typical insertion procedures involve advancing the distal portion of the device by rotational movement into the vitreous of the eye. In many cases, in order for the device to be advanced into the vitreous, it is first advanced through a scleral region, or scleral and conjunctival regions of the eye. In these aspects, advancing the device into these types of tissues and/or body materials can involve a process that is more delicate than that of advancing a device into a dense and hardened tissue, such as osseous tissue.

The vitreous cavity is the largest cavity of the eye and contains the vitreous humour or vitreous. In reference to FIG. 2, the vitreous is bound interiorly by the lens, posterior lens zonules and ciliary body, and posteriorly by the retinal cup.

The vitreous is a transparent, viscoelastic gel which is 98% water and has a viscosity of about 2-4 times that of water. The main constituents of the vitreous are hyaluronic acid (HA) molecules and type II collagen fibers, which entrap the HA molecules. The viscosity is typically dependent on the concentration of hyaluronic acid within the vitreous. The vitreous is traditionally regarded as consisting of two portions, one, a cortical zone, characterised by more densely arranged collagen fibrils, and two, a more liquid central vitreous. The vitreous can be further subdivided for descriptive purposes into three major topographical zones, namely preretinal, intermediate, and retrolental zones, and by two tracts, the preretinal and retrolental tracts. Local variations in vitreal anatomy most likely reflect small variations in the density of the constituents, namely vitreous tracts being condensations of collagen and more liquid areas being richer in soluble proteins and hyaluronic acid.

Therefore, in other aspects, the invention provides an insertion instrument and method for rotatably inserting a device into a target region of the body, the target region comprising a gel-like material, such as viscoelastic gel.

In many aspects of the invention, ocular insertion is performed by rotatably inserting at least a part of, and typically, most of the device into the vitreous. In some aspects, the device can be driven through the scleral tissue through a penetration in the scleral tissue (trans-scleral insertion) caused by a sharp distal end of the device. Alternatively, in other aspects, the device can be driven into vitreous through a sclerotomy previously made in the eye.

In many cases, as indicated, the device it is first advanced through a scleral region of the eye. The sclera forms the principal part of the outer fibrous coat of the eye and functions to both protect the intraocular contents and maintain the shape of the globe when distended by intrinsic intraocular pressure (IOP). The sclera is relatively avascular and generally appears white externally. The viscoelastic nature of the sclera (great tensile strength, extensibility and flexibility) allows only limited distension and contraction to accommodate minor variations in IOP. The sclera includes connective tissue comprising primarily of collagen (mostly types I and III). The sclera is thickest posteriorly (1 mm) and thinnest (0.3-0.4 mm) behind the insertions of the aponeurotic tendons of the extraocular muscles. It is covered by the fascia bulbi posteriorly and conjunctiva anteriorly. The three histological layers of the sclera are the lamina fusca, stroma and episclera.

Therefore, in other aspects, the invention provides an insertion instrument and method for rotatably inserting a device through tissue or membrane comprising connective tissue or tissue that includes collagen as a primary component. In another aspect, the tissue or membrane can have a thickness of in the range of about 0.2 mm to about 1.0 mm.

What is claimed is:

1. A insertion instrument for rotatably inserting an insertable medical device into a portion of the eye, the instrument comprising:
a proximal portion and a distal portion, the distal portion comprising a securing member capable of engaging an insertable medical device and holding the device in position for rotational insertion into a portion of the eye, and
an actuating mechanism for releasing the insertable medical device from the securing member.

2. The insertion instrument of claim 1 for rotatably inserting an insertable medical device into a posterior portion of the eye

3. The medical insertion instrument of claim 1 wherein the securing member comprises two or more radially contractible and expandable fingers.

4. The medical insertion instrument of claim 3 wherein the securing member comprises four or more radially contractible and expandable fingers.

5. The medical insertion instrument of claim 3 wherein the two or more radially contractible and expandable fingers have distal ends comprising lips.

6. The medical insertion instrument of claim 3 wherein the fingers, when in a contracted state, form at least a portion of a socket having a geometry that accommodates the proximal portion of the insertable medical device.

7. The medical insertion instrument of claim 6 wherein the socket has a concave shape.

8. The medical insertion instrument of claim 6 wherein the periphery of the socket has a rounded shape.

9. The medical insertion instrument of claim 6 wherein the socket has a volume of 5 mm³ or less.

10. The medical insertion instrument of claim 3 comprising a housing having a bore wherein the fingers, when in a contracted state, are at least partially disposed within the bore.

11. The medical insertion instrument of claim 1 wherein the distal end of the insertion instrument comprises a tapered shape.

12. The insertion instrument of claim 3 wherein the actuating mechanism causes extension of the two or more radially contractible and expandable fingers in a direction parallel to the axis of the instrument and from the distal end of the instrument.

13. The insertion instrument of claim 1 the movement of the fingers caused by the actuating mechanism is self-retractable.

14. The insertion instrument of claim 3 wherein the fingers are formed from a common piston having a piston axis.

15. The insertion instrument of claim 1 comprising a housing comprising a bore and a piston disposed within the bore, wherein the piston is independently rotatable from the housing about a first axis, and that is in rotational communication with the securing member.

16. The insertion instrument of claim 15 comprising a housing comprising a sliding member movable in at least one direction along the axis of the instrument, wherein movement of the sliding member causes rotation of the piston and corresponding rotation of the securing member.

17. The insertion instrument of claim 16 wherein both the sliding member and the piston comprise threads, which are meshed to form a gearing pair.

18. The medical insertion instrument of claim 1 wherein the securing member comprises a surface that has been treated to increase the coefficient of friction between said surface and proximal portion of the insertable medical device.

19. The medical insertion instrument of claim 1 comprising a vacuum chamber capable of being actuated by the actuating mechanism, and in gaseous communication with the securing member.

20. A kit for rotatably inserting an insertable medical device comprising a non-linear shape into a portion of the eye, the kit comprising:
an insertable medical device comprising a distal portion having a non-linear shape and a proximal portion comprising a head, and
an instrument comprising:
a proximal portion and a distal portion, the distal portion comprising a securing member capable of engaging the head of the insertable medical device and holding the device in position for rotational insertion into the eye, and
an actuating mechanism for releasing the insertable medical device.

21. The kit of claim 20 wherein the insertable medical device is pre-loaded in the securing member.

22. A method for rotatably inserting a medical device into a portion of an eye, the method comprising the steps of:
providing an insertion instrument comprising a proximal portion and a distal portion, the distal portion comprising a securing member and a medical device engaged by the securing member;
rotatably inserting the medical device into the eye; and
triggering an actuating mechanism to release the medical device from the securing member.

23. The method of claim 22 where, in the step of providing and rotatably inserting, the securing member comprises two or more fingers that are in a radially contracted state and that engage a proximal portion of the medical device, and in the step of triggering the fingers are radially expanded to disengage and release the proximal portion of the device.

24. The method of claim 23 where, in the step of providing and rotatably inserting, the fingers in the radially contracted state form at least a portion of a socket having a geometry that accommodates the proximal portion of the insertable medical device.

25. The method of claim 23 where the insertion instrument comprises a housing comprising a bore extending from the distal end of the housing, and in the steps of providing and rotatably inserting, the fingers are in a radially contracted state and are at least partially disposed within the bore at the distal end, and, in the step of triggering, the fingers extend from the distal end of the bore, thereby allowing the fingers to expand radially.

26. The method of claim 22 where the device comprises a housing that is in rotational communication with the securing member, and in the step of rotatably inserting, the housing is rotated to drive the insertable medical device into the eye.

27. The method of claim 22 where the device comprises a housing, a bore within the housing, and a piston disposed within the bore, wherein the piston is independently rotatable from the housing about a first axis but in rotational communication with the securing member, where, in the step of rotatably inserting, the piston is rotated about the first axis to cause rotational insertion of the medical device into the eye.

28. The method of claim 27 wherein the housing comprises a sliding member in physical communication with the piston, and in the step of rotatably inserting, the sliding member is moved in at least one direction along the first axis, causing rotation of the piston and corresponding rotation of the securing member.

29. The method of claim 22 where, in the step of rotatably inserting, the medical device is engaged in the securing member by suction that is applied to the proximal end of the medical device.

30. The method of claim 22 where, in the step of rotatably inserting, the insertable medical device is driven through the sclera.
